(19) United States
(12) Patent Application Publication
(10) Pub. No.: US 2006/0180479 A1
(43) Pub. Date: Aug. 17, 2006

(54) Method, system and device for obtaining electrochemical measurements

(76) Inventors: Douglas I. Sparkes, Kitchener (CA); Liam O'Hagan, Walkerton (CA); Peter Ertl, Vienna (AT); Kirk Miller, Waterloo (CA); Thomas Stephen Mann, Hanover (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(21) Appl. No.: 11/339,486

(22) Filed: Jan. 26, 2006

Related U.S. Application Data
(60) Provisional application No. 60/646,640, filed on Jan. 26, 2005.

(51) Int. Cl. G01F 1/64; G01N 33/487; G01N 27/26

(52) U.S. Cl. 205/792; 204/403.01; 204/406

(57) ABSTRACT

The invention is directed at apparatus for high-speed acquisition of electrochemical measurements from multiple biochemical or microbiological samples comprising an array of electrodes; a voltage signal generator for the array of electrodes; and means for collecting electrochemical measurements from the electrodes; wherein when the electrodes are brought in contact with the multiple biochemical or microbiological samples, the voltage signal generator provides a voltage to each of the electrodes to produce the electrochemical measurements for the means for collecting to retrieve.

### CROSS REFERENCE TO RELATED APPLICATIONS

This application claims the benefit of U.S. Provisional Application No. 60/646,640, filed Jan. 26, 2005, which is incorporated herein by reference.

### FIELD OF THE INVENTION

This invention relates to the field of parallel electrochemical testing. In particular, the invention finds use in monitoring assays contained within various test formats, including, but not limited to microtiter plates, miniaturized test panels and petri plates.

### BACKGROUND OF THE INVENTION

Many conventional systems exist for performing tests using single-measurement systems while the application of electrochemical techniques is used in a variety of scientific fields. Electrochemical instrumentation is relatively inexpensive and is generally perceived as a very sensitive analysis technique. Although electrochemical analysis methods carry advantages such as the absence of colour and turbid interferences over spectroscopic methods, electrochemical parallel measurement systems have not become widely available in the scientific community.

In the last decade several multi-channel analysis systems have been used where a multitude of home-made electrodes were connected to commercially-available potentiostats via relay boards or multiplexers. The integration of these electrodes (e.g. 8 and 16) with existing instrumentation was aimed at creating sequential electro-analysis systems. Although the application of various electrochemical analysis techniques such as CV, DVPV etc. were made possible limitations of these hybrid configurations included their complex configuration, and cumbersome analysis set-up which resulted in low reproducibility, external noise interferences and limited reliability of electrodes.

More recently an electrochemical oxygen biosensor using a 96-electrode format was employed in a study that investigated the cytotoxic effects of isoflavonoids on cancer cells. The system was equipped with 12 disposable substrates each containing three screen-printed electrodes for any of the 8 electrochemical cells located at each substrate. Although the ability to perform multiple parallel measurements has been demonstrated using the amperometric oxygen sensor serious limitations still exist including low reproducibility and repeatability exhibiting precision of approx. 20% (RSD) between measurements.

Other examples of prior art systems include:

U.S. Pat. No. 6,247,350 to Tsukada et al. describes an electrochemical sensor capable of measuring dissolved oxygen in 96 test samples. The system is equipped with a multipotentiostat connected to a sensor array comprising of 12 disposable substrates containing three screen-printed electrodes for each of the 8 electrochemical cells located on each substrate. The disposable screen-printed microelectrodes are modified using a gold plating procedure.

Limitations of this configuration include precision and reproducibility associated with the variability of the disposable electrodes. This device has been used to measure amperometrically dissolved oxygen in solution and has been applied to monitor microbial respiratory activity via the consumption of dissolved oxygen. In addition, any problems such as bad contacts or corrosion phenomena occurring at the connection site between the disposable substrates and the connector to the electronics system cause a total loss of signal.

U.S. Pat. No. 6,649,402 to Van der Weide et al. describes a microfabricated multiwell apparatus that allows rapid microbial growth assays by measuring the capacitance or resistance or both between the electrodes at each well. In this invention, a commercially available meter capable of measuring capacitance, resistance or inductance, is connected to a switch/control unit. The switch/control unit sequentially connects the meter to the electrodes of one selected well. Although this invention applies a two-electrode system, it is not considered a controlled-current technique since it measures the mobility of ions in solution rendering its application to a narrow analytical field. Using impedance measurements, only changes to the overall composition of the solution can be detected, but it does not detect single analytes or electroactive species in the test sample.

U.S. Pat. No. 6,235,520 to Malin et al. describes a high-throughput screening method and apparatus that measures conductance changes across two electrodes of a test sample. This apparatus has been used to monitor the level of growth or metabolic activity of microbial cells contained in each well. A small alternating AC voltage is applied and a multiplexing or sampling circuitry interrogates sequentially each microwell by applying a short duration signal to each well, measuring the current across the “stimulated” electrodes.

U.S. Pat. No. 5,312,590 to Gunasingham describes an amperometric sensor for single and multicomponent analysis. This device includes multiple sensing elements each coated with perfluorinated ion-exchange polymer film incorporating a redox mediator; an immobilized enzyme layer and, over this, a semipermeable membrane. The technique proposed in the invention is particularly suitable for the determination of glucose and cholesterol in biological fluids. The device consists of four symmetrically arranged sensor elements that enable multi-species determination using a single test sample. Each sensor element is coated with a unique reaction layer that makes it responsive to specific chemical species.

It is, therefore, desirable to provide a novel system, method and device for obtaining electrochemical measurements.

### SUMMARY OF THE INVENTION

The invention provides easy-to-use, adaptable, and convenient solutions for an instrument that monitors assays electrochemically, especially multiwell assays using a high speed data acquisition system.

This device is, preferably, used for the electrochemical analysis of solutions or liquid suspensions by two-electrode amperometric methods including chronoamperometry, chronocoulometry and biamperometry. In one embodiment, the device allows parallel simultaneous experiments on 48 samples present in the wells of a multiwell plate. The device applies a constant voltage between two electrodes immersed in each well, and measures current flowing between the two electrodes over a period of time. Current may be integrated to present total charge as a function of time. For chronoamperometry and chronocoulometry, the two electrodes are made of different materials (e.g. platinum, gold or silver) while the biamperometry method uses electrodes made of the same material (e.g. platinum, gold etc.). This device may be applied to the analysis of chemical sample components (e.g. ascorbic acid), enzymes (e.g. glucose oxidase or peroxidase), immunoassay or binding assay labels (e.g. a biotin-peroxidase label in a biotin assay), and viable cells (microorganisms, plant cells, animal cells).

The invention provides an analysis system for performing highly reliable, precise and accurate electrochemical measurements using a low-noise and a high-speed sequential data acquisition system. In addition, the robust sensor design includes an array of identical electrodes allowing for a high degree of reproducibility between multiple measurements. In summary, the invention performs measurements of an analyte using a biamperometric analysis technique such as measuring changes in current or charge over time.

The invention provides an analysis system that combines the advantages of electrochemical detection with simultaneous parallel measurements using a reusable sensor array. In particular, the invention provides high-speed sequential data acquisition system that tests a plurality of multiple-well test panels. In addition, the described embodiment performs both endpoint detection and kinetic investigations of reduced or oxidized electro-active species in solution. Moreover, the electronic system analyzes the gathered test data to produce accurate and reproducibly information about the concentration of each redox-active compound in the test wells.

A re-usable sensor design is best suited to maintain stable and consistent electrical contacts between electrochemical cells and the data acquisition system. The robust design of the invention thus allows for simple instrumentation and measuring conditions, high sensitivity, high selectivity, and a high signal-to-noise ratio. In one embodiment, the invention comprises a multilayered electronics board that is directly connected to individually addressable electrodes. As a result of the close proximity between the associated electronic components and the electrochemical cells reliable data collection is performed in a low noise environment. In another embodiment the developed re-usable sensor array comprises, but is not limited to, 48 electrochemical cells (studs) each containing two solid platinum electrodes of identical shape and size. The electrodes are, preferably, embedded in a non-wetting insulating material and are located near the tip of the stud in order to establish optimum electrical paths during measurement. The three-dimensional studs are further designed to eliminate bubble formation or entrapment during fluid penetration.

In an aspect of the invention, there is provided apparatus for high-speed acquisition of electrochemical measurements from multiple biochemical or microbiological samples comprising an array of electrodes; a voltage signal generator for the array of electrodes; and means for collecting electrochemical measurements from the electrodes; wherein when the electrodes are brought in contact with the multiple biochemical or microbiological samples, the voltage signal generator provides a voltage to each of the electrodes to produce the electrochemical measurements for the means for collecting to retrieve.

In another aspect, there is provided a method of obtaining electrochemical measurements from multiple biochemical or microbiological samples comprising the steps of generating a voltage; applying the voltage to a plurality of electrodes; and retrieving electrochemical measurements from the electrodes after the plurality of electrodes contact the multiple samples.

### BRIEF DESCRIPTION OF THE DRAWINGS

These and other aspects, features and advantages of the invention can best be understood by reference to the detailed description of the preferred embodiments set forth below taken with the drawings in which:

FIG. 1a is a schematic diagram of a first embodiment of apparatus for obtaining electrochemical measurements using a high speed data acquisition system;

FIG. 1b is a schematic diagram of an embodiment of an electronics board of the apparatus of FIG. 1a;

FIG. 2a is a flowchart outlining a first embodiment of a method of high speed acquisition of electrochemical measurements;

FIG. 2b is a flowchart outlining an embodiment of measurement gathering;

FIG. 3 is a perspective view of a plate containing solutions to be tested;

FIG. 4 is a schematic view of a sensor array of the apparatus of FIG. 1a;

FIG. 5 is a front perspective view of a second embodiment of apparatus for electrochemical measurements using a high speed data acquisition system;

FIG. 6a is a schematic diagram of a first embodiment of a printer circuit board (PCB) and electrochemical cells mounted to the sensor array;

FIG. 6b is a second embodiment of the PCB and electrochemical cells mounted to the sensor array;

FIGS. 7a and 7b are perspective views of a method of manufacturing the embodiment of FIG. 6a

FIGS. 8a and 8b are perspective views of a method of manufacturing the embodiment of FIG. 6b

FIG. 9 shows an arrangement of an electrochemical cell;

FIGS. 9a to 9d show further embodiments of electrochemical cells;

FIGS. 10a to 10f shows various shapes and sizes of electrode tips;

FIG. 11 is an embodiment of a reusable electrochemical cell; and

FIGS. 12a and 12b show data using the oxidation and reduction reaction of ferricyanide/ferrocyanide redox couple.

### DETAILED DESCRIPTION OF THE PREFERRED EMBODIMENTS

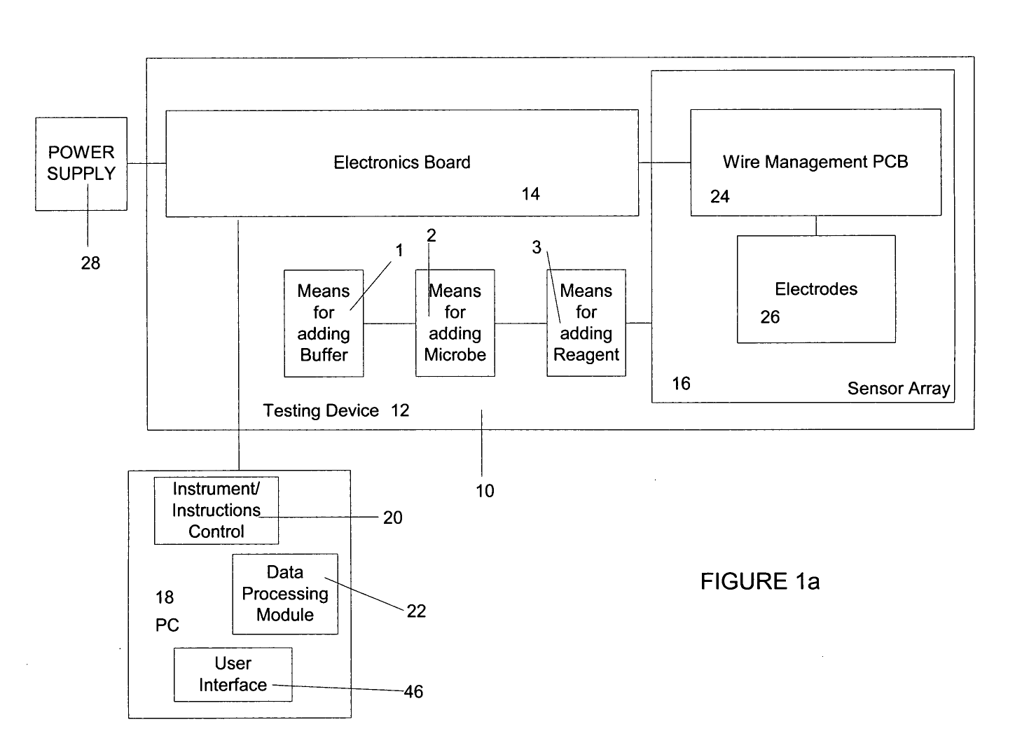

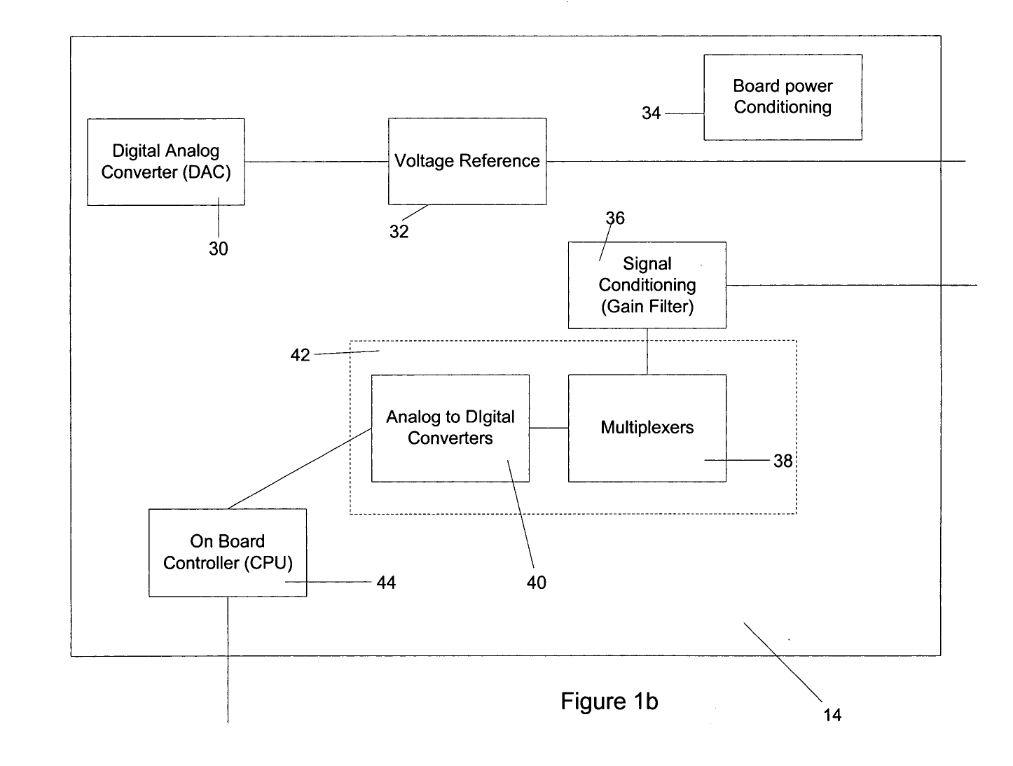

Turning to FIG. 1a, a schematic diagram of apparatus for high-speed data acquisition of electrochemical measurements of multiple biochemical or microbiological samples is shown. It will be understood that the apparatus may also be used for electrochemical measurements of chemical solutions or for other bio-analytical measurements. The apparatus 10 comprises a testing device 12 having an electronics board 14, including hardware for receiving and transmitting signals, and a sensor array 16. A more detailed diagram of an embodiment of the electronics board 14 is shown in FIG. 1b. The testing device 12 may include an internal power supply or power may be supplied to the testing device 12 from an external power source 28.

The electronics board 14 is connected to a computer (PC) 18 along with the sensor array 16. The PC 18 preferably comprises means (such as a software module) to transmitting instructions (in the form of signals) 20 to the electronics board 14 to control operation of the apparatus 10 along with means (such as a software module) for processing data 22 received from the electronics board 14 as a result of electrochemical measurements. A user may interact with the PC 18 (and thereby the apparatus 10) via a user interface module 46. The sensor array 16 comprises a wire management printed circuit board (PCB) 24 and a set of electrodes 26.

In the present embodiment, the testing device 12 also includes a means for adding a buffer 1, a means for adding a microbe 2 and a means for adding a reagent 3. Each of these means for adding 1, 2 and 3 are used for mixing with the samples in order to prepare the samples for testing. It will be understood that this process is preferably automated so that the testing process may be accelerated in order to gain full advantage of the high speed data acquisition. However, it will be understood that the buffer, microbe and reagent may also be added manually rather than being automated as in the present embodiment. The sensor array 16 is preferably housed in a shielded enclosure to protect the sensor array 16 from electromagnetic interference.

Turning to FIG. 1a, the electronics board 14 preferably comprises a digital-to-analog converter (DAC) 30 (which generates a fixed voltage or an arbitrary voltage waveform) resulting in a voltage reference 32, which in turn, is connected to the PCB 24 of the array 16. The DAC 30 preferably includes a feedback mechanism for verifying that the generated voltage matches an expected value. The voltage reference 32 is responsible for applying a voltage (or current) to all the electrodes 26 in the sensor array 16. The voltages (or currents) applied to the cells can be adjusted to a predetermined setting. The voltage (or current) may comprise of DC components, AC components, or both. In the preferred embodiment, a DC voltage is applied to all electrodes. The voltage reference 32 ensures a stable, accurate voltage level is delivered to the individual electrodes.

A board power conditioning system or device 34 is also located on the electronics board 14. The system 34 is responsible for providing clean and stable power to the other parts of the electronics board 14. In addition to power conditioning and regulation, the system preferably includes surge protection for protecting the electronics on the electronics board 14. Further, adequate heat sinking is provided to ensure that the electronics boards do not over heat. In the preferred embodiment, the power conditioning system 34 includes several voltage regulators to ensure on-board voltages are stable and maintain appropriate levels.

A signal conditioning means, preferably amplifications and/or filtering, 36 is also connected to the sensor array 16 and to a set of multiplexers (MUX) 38 and analog-to-digital converters (ADC) 40 which may be combined to form a means for converting analog signals to digital signals 42. In the preferred embodiment, multiplexing is added to reduce the number of ADCs 40 such that the multiplexer 38 connects a selected current signal to one of the ADC 40.

The signal conditioning means 36 is preferably responsible for measuring and processing the measured current (or voltage) signals from the sensor array 16. This may include amplifying, filtering, and digital sampling. In the present embodiment, current signals from the electrodes are amplified and digitally sampled by one of the ADC 40, operating at a high sampling rate. Each of the electrodes 26 is measured sequentially but the sampling and switching is so fast in comparison to the signal it is sampling that it could be said that measurements are made in parallel. The method of data acquisition will be explained in more detail below.

The means for converting analog signals to digital signals 42 is connected to an on board controller, or CPU, 44 which is, in turn connected for communication with the PC 18.

Prior to the testing of the samples to obtain electrochemical measurements, the apparatus 10 is turned on such that power is supplied (via the power supply 28 in the current embodiment) to the electronics board 14. The CPU 44 receives instructions from the instruments control 20 of the PC 18 (preferably entered by a user via the user interface 46) which then transmits signals to the DAC 30 to convert the voltage reference parameter entered via the user interface into a reference voltage. The generated voltage signal from the DAC 30 becomes the voltage reference 32 for each of the electrodes 26 in the sensor array 16 for use in the electrochemical measurements. As described above, the board power conditioning system 34 preferably continuously monitors the current and voltage levels of all the parts of the electronics board 14 to verify that all of the parts are operational.

In the present embodiment, the user interface 46 within the PC 18 allows a user to determine and control the voltage being supplied to the sensor array 16 along with determining the format in which the acquired data is processed.

After the voltage/analog signal is transmitted to the sensor array 16, the sensor array 16 collects the required signals in order to obtain separate electrochemical measurements, such as a current reading, from each of the electrodes 26 as described below. The electrochemical measurements (in an analog form) are then transmitted back to the electronics board 14, and more specifically, to the signal conditioning means 36 which acts as a gain and/or filter to the received signals. The filtered signal is then transmitted to the set of multiplexers (MUX) 38 and analog-to-digital converters (ADC) 40 which then converts the electrochemical measurements from analog signals to digital signals. Operation of the set of MUX 38 and set of ADC 40 will be understood by one skilled in the art. Furthermore, although only one set of MUX/ADCs are shown, it will be understood that multiple sets may be provided which allows multiple sensor arrays to be connected to a single electronics board 14.

After the signals are converted, they are transmitted to the CPU 44 which then forwards the measurements (in digital form) to the PC 18. After receiving the measurements, the data processing module 22 of the PC 18 processes the measurements in order to display the information requested by the user. The displayed information is preferably calculated as a function of the analog current measurements obtained by the electrodes. After the data is processed (in accordance with the user's instructions), the information is displayed to the user.

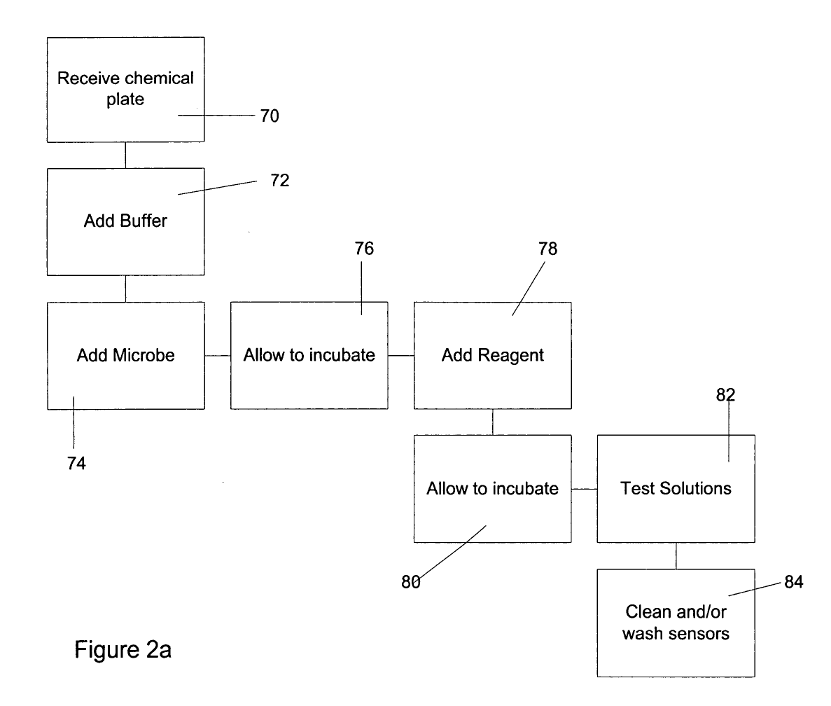

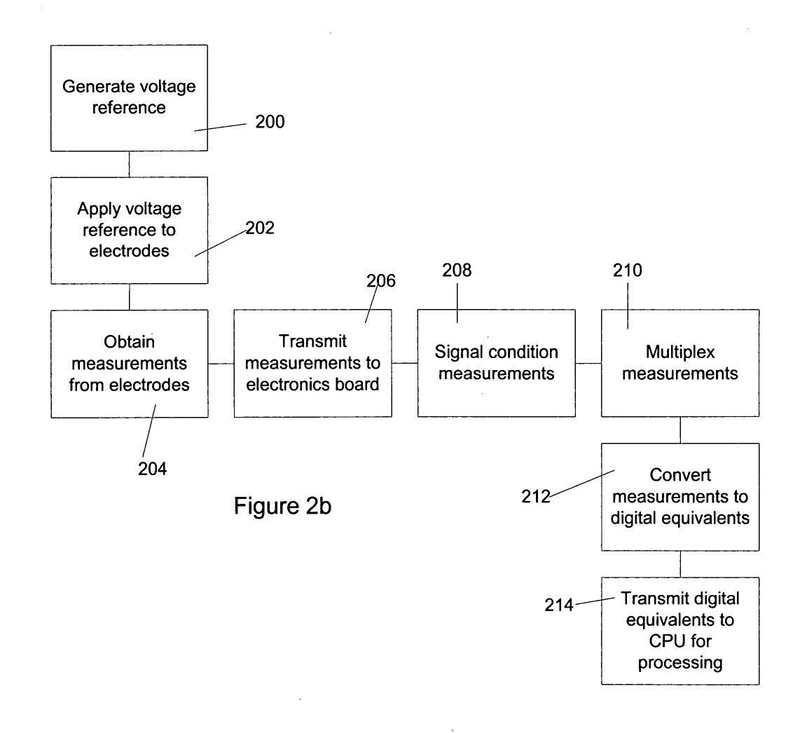

As shown in FIG. 2, a first embodiment of a method of high-speed acquisition of electrochemical measurements is shown.

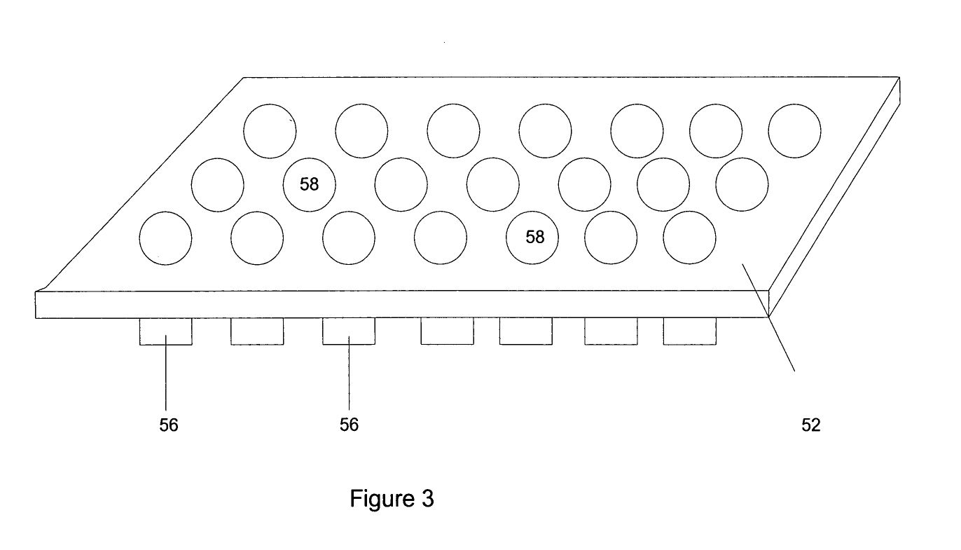

After the chemicals to be tested are received (step 70), typically in a plate 52 (as schematically shown in FIG. 3), a buffer is preferably added to each of the wells 56 via the openings 58 (step 72) which may be performed manually or via the means for adding buffer 1. The combination of an electrode 26 and one of the wells 56 form an electrochemical cell. As will be understood, the number of wells may be greater than or equal to the number of electrodes 26 in the array 16. In a microbiological testing application, after the buffer is added, a microbe is preferably added to each of the wells 56 (step 74). Again, this may be performed manually but is preferably performed automatically by the means for adding microbe 2. After these two ingredients are added to the samples, there is preferably an incubation period (step 76) of preferably 10 minutes allowing the samples to react/be exposed to the buffer and the microbe. Although 10 minutes is preferred, the period of incubation may be as low as 30 seconds or as high as a few hours. After this incubation period, a reagent such as ferrocyanide is added to the wells 56 (step 78). Other reagents include ferricyanide (hexacyanoferrate (III)); dichlorophenol-indophenol (DCIP); ferrocene and ferrocene derivatives; methylene blue; janus green; tris(bipyridyl)iron (III); a quinone; or a phenazine. When the mediator is a quinone, specific examples include: benzoquinone, naphthoquinone, menadione, anthraquinone, or any substituted derivatives of these. When the mediator is a phenazine, specific examples include: phenazine methosulfate or phenazine ethosulfate. Along with the reagents, Effectors or effector compounds are reagents may also be used which provide an impact on the reagent. Example effectors include glucose, lactic acid, arginine, pyruvate, nitrate, D-mannose, succinate, L-tryptophan, sucrose, D-fructose, D-galactose, formic acid, L-lysine, D-sorbitol, D-lactose, beta-cyclodextrin, alpha-ketoglutarate, citric acid, D-xylose, D-arabinose, malonic acid, L-rhamnose, L-ornithine or beta-glycerophosphate. After the reagent is added, there is preferably another incubation period (preferably of 10 minutes) (step 80) in which the samples are allowed to react/be exposed to the reagent and/or effector. In both of the incubation periods, the temperature at which the incubation occurs is preferably between 20 and 50 degrees Celsius and more specifically between 30 and 40 degrees Celsius. However, it will be understood that the incubation period may occur at higher or lower temperatures. The temperature range differs depending on the nature of the samples.

After the second incubation period (step 80), the plate 52 is then inserted into the sensor array 16. The electrodes 26 are then lowered into each of the wells and the voltage applied to each of the electrodes 26. As the voltage is being applied, via the electrodes 26 to the solution in the wells, electrochemical measurements (such as current) are taken from each of the wells in a predetermined manner (step 82) (thereby rendering the measurements virtually parallel) and then transmitted to the electronics board 14 whereby the measurements are converted to digital signals for processing by the PC 18.

FIG. 2b provides one embodiment of testing performed in step 82. After the second incubation period, a voltage is generated by the DAC 30 (step 200). After generating the voltage, the voltage is transmitted and applied to the electrodes (step 202). After the voltage is applied and the electrodes are contacting the solutions to be tested, electrochemical measurements are obtained through the electrodes (step 204). These measurements are then preferably gathered by the PCB 24 and transmitted to the electronics board 14 (step 206). The electrochemical measurements are then preferably signal conditioned such as by applying a gain and/or a filter to the measurements (step 208). The measurements may then be multiplexed (step 210) depending on the number of samples being tested. After being multiplexed, the measurements are converted from an analog signal to its digital equivalent (step 212). After converting the measurements, the digital equivalents are transmitted to a CPU (such as the on board controller 44 or CPU 18) for processing of the electrochemical measurements (step 214).

It will be understood that the testing period and the testing cycles are preferably determined by the user such that the voltage is applied to the electrodes for the predetermined time period. As long as a voltage is being applied to the sensor array, the sensor array 16 continues to measure the current in each well and transmits this information to the electronics board 14. After the measurements are completed, the plate is removed and the electrodes cleaned and/or washed (step 84) so that they the sensor array 16 is ready for the next set of measurements. In an alternative embodiment, the electrodes 26 may be for one-time use in which the electrodes 26 are then removed and a new set of electrodes mounted to the sensor array 16.

As outlined above, during the electrochemical measurement acquisition, the timing between readings (testing cycle) is determined by the user via the user interface 46 of the PC 18.

Although shown as being separate from the testing device 12, it will be understood that the contents of the PC 18 may be a part of the testing device 12. However, in the preferred embodiment, the PC 18 is external so that the testing device 12 may be portable and connected to any PC which includes the necessary instruction, or instrument, control 20, data processing module 22 and user interface 46.

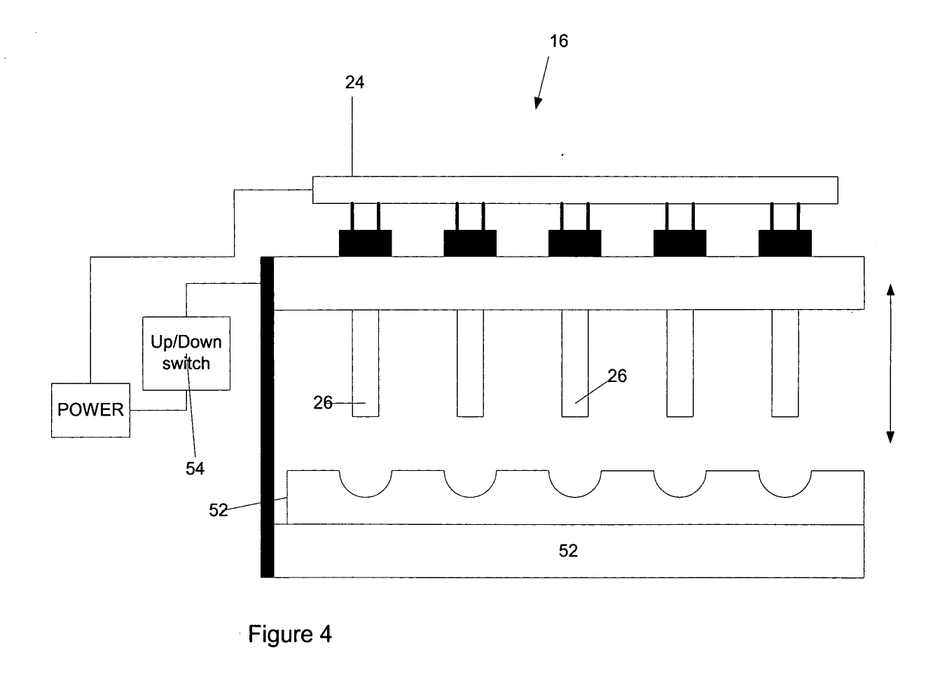

FIG. 4 is a schematic diagram of the sensor array 16. As disclosed above, the sensor array 16 comprises the PCB 24 and the set of electrodes 26. In this embodiment, at the bottom of the sensor array 16, is a housing 50 for receiving the assay plate 52 storing the biochemical or microbiological samples to be tested. Alternatively, the plate may simple rest on a platform. In this figure, it is assumed that the assay plate 52 has already passed by the means for adding a buffer 1, the means for adding a microbe 2 and the means for adding a reagent 3 so that the samples are ready for testing. Alternatively, the buffer, microbe and reagent may be added after the plate 52 has been placed in the housing 50 either via automation or manually. When the testing is to commence, the electrodes 26 are lowered from a raised position and placed in contact with the solution in the wells of the plate. The positioning of the electrodes is preferably achieved by activating a device such as a switch 54 which controls the position of the electrodes. Alternatively, this may be performed manually.

After the electrodes 26 are in contact with the samples, the voltage is supplied via the electronics board 14 (through the PCB 24) and then to each of the electrodes 26. After the voltage is provided, the electrodes retrieve electrochemical measurements, such as current, which are then transmitted back to the signal conditioning device 36 in the electronics board (via the PCB 24).

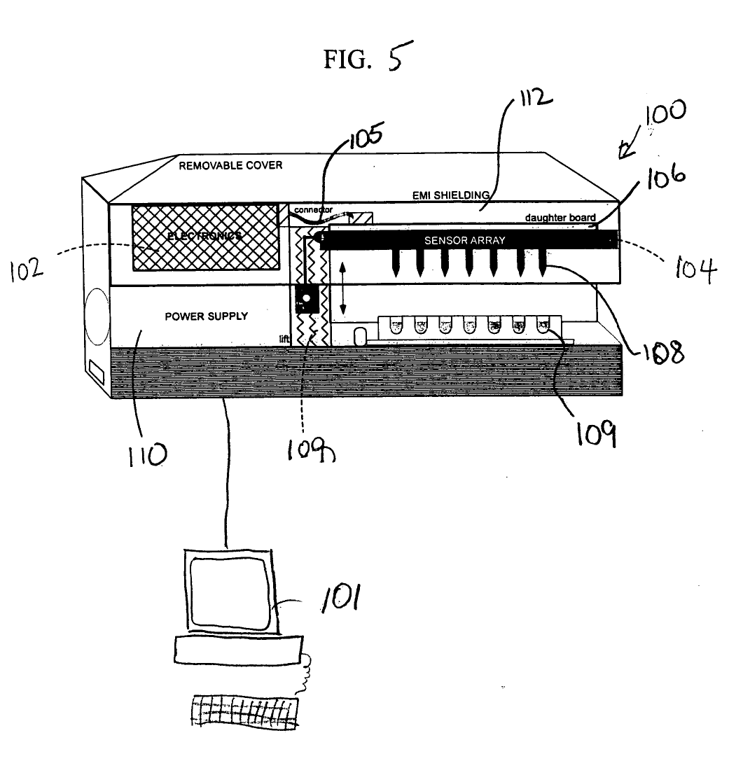

FIG. 5 is a schematic diagram of another embodiment of apparatus for obtaining electrochemical measurements.

The apparatus 100 comprises an electronics section 102 including an electronics board (not shown) as described above. The electronics section 102 is connected to a sensor array 104 (via a cable or connector 105) which comprises a printed circuit board 106 and a set of electrodes 108. A means for moving the electrodes 109 towards and away from the biochemical and/or microbiological samples is provided. The apparatus 100 also includes a power supply 110 along with a user interface (not shown) allowing a user to interact with the apparatus 100 to define data collection and processing parameters and the voltage level (or waveform) at which the samples are being tested. Alternatively, the apparatus 100 may be connected to a computer 101 which controls the operation of the apparatus 100 (in a manner similar to the one described above). The sensor array 16 is preferably located within a shielded enclosure 112 to protect the readings from electromagnetic interference.

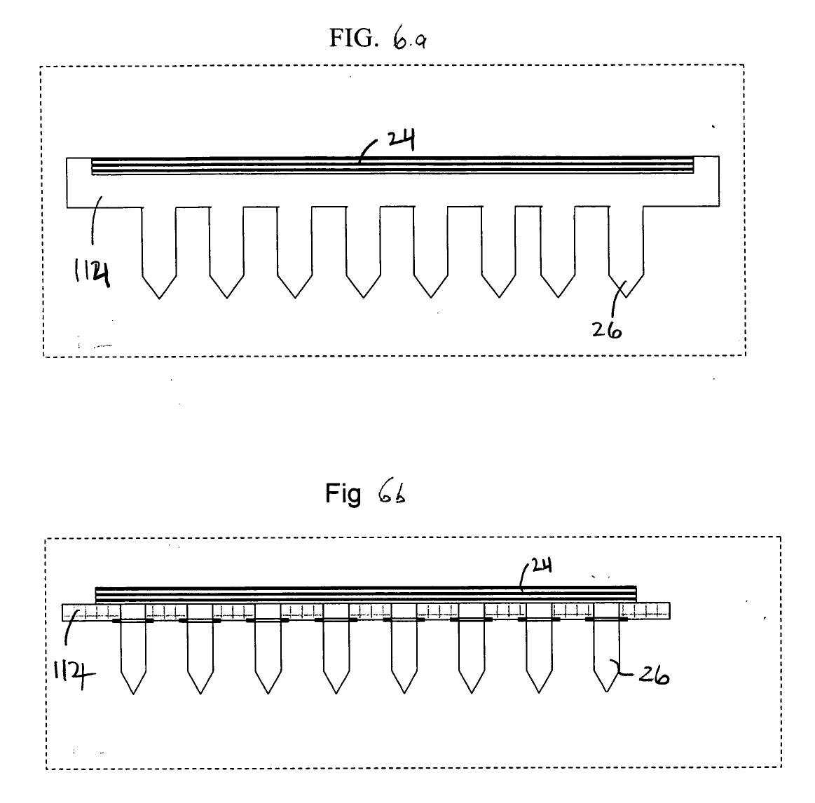

FIGS. 6a and 6b provide two examples of how the electrodes and PCB are mounted in the sensor array.

In FIG. 6a, the electrodes 26, along with a sensor array base 114, form a one-piece compact sensor apparatus. The PCB 24 is located within the sensor array base 114 and in communication with each of the electrodes 26 to provide the necessary voltage to and obtain the electrochemical measurements from the electrodes 26. In this manner, the electrodes 26 may be quickly and easily replaced when required since all that is required is to simply exchange the existing sensor array base 114 with a new one.

In FIG. 6b, the electrodes 26 are connected individually within a sensor array base 114. The PCB 24 is located atop the sensor array base 114 in direct connection with the electrodes 26 in order to provide the necessary voltages to and obtain the electrochemical measurements from the electrodes 26. In this example, when an electrode becomes defective, the individual electrode may simply be replaced without having to replace the entire array.

In both of these examples, the electrodes 26 are pencil shaped electrochemical cells containing indicator electrodes (not shown) and designed to minimize the potential for bubble formation during fluid penetration. Although only eight electrodes are shown in both FIGS. 6a and 6b, it will be understood that an array of electrodes are present as will be shown with respect to FIGS. 7 and 8. In the preferred embodiment, the sensor array comprises of 96 electrodes, but it may contain any other number of a multiple of 2 (e.g. 8, 16 . . . 128 etc).

Turning to FIGS. 7a and 7b, perspective views of the sensor array shown in FIG. 6a are provided. FIGS. 7a and 7b show assembly drawings of part of the sensor array comprising a compact reusable electrode array design. In this embodiment, the “mold” design has been used to build a compact sensor array 16 comprising a solid block featuring 48 electrodes. FIG. 7a shows a bottom view of the sensor array having pencil shaped electrodes 26 and PCB 24. It will be understood that the PCB may be replaced by the electronics board.

This embodiment is designed to minimize possible corrosion phenomena at contact points between the electrodes 26 and leads, or indicator electrodes as well as the PCB 24 and for applications at higher temperature settings (evaporation issues) or for the investigation of corrosive test samples. FIG. 7b shows the assembly drawing of the sensor array 16 comprising connection points at the PCB 24 and low-resistance leads 113. At the sampling end of the electrodes 26 are two, preferably Pt, indicator electrodes. In this configuration, the electronics board or wire management board 24 is integrated into top of the sensor array base. An additional insulating material such as a Silicon-layer may be applied between the electronics board/PCB and the sensor array base 114.

Turning to FIGS. 8a and 8b, perspective views of the sensor array shown in FIG. 6b are provided. In this embodiment individually manufactured electrodes 26 are directly connected to the electronics board 14 or indirectly via the wire management board (PCB) 24. As shown in FIG. 8b, each of the electrodes 26 are mounted within the sensor array base 114, via an electrode support 116 with two leads 118 which are preferably soldered to the PCB 24. With this embodiment, electrodes 26 may be individually removed and exchanged. In addition various electrode materials may be applied to a predetermined number of electrodes on the same sensor array design. This design allows for the simultaneous investigation of or with different electrode materials.

In embodiments where there are less than 48 electrodes, the electronics board 14 may be located within the sensor array 16 such at the electrodes 26 are connected directly to the electronics board 14. However, in the case of higher density sensor arrays, e.g. 48 or greater electrodes, the data acquisition electronics board is preferably housed in a separate shielded enclosure with a wire management board (PCB) located in the sensor array for communication with the electronics board.

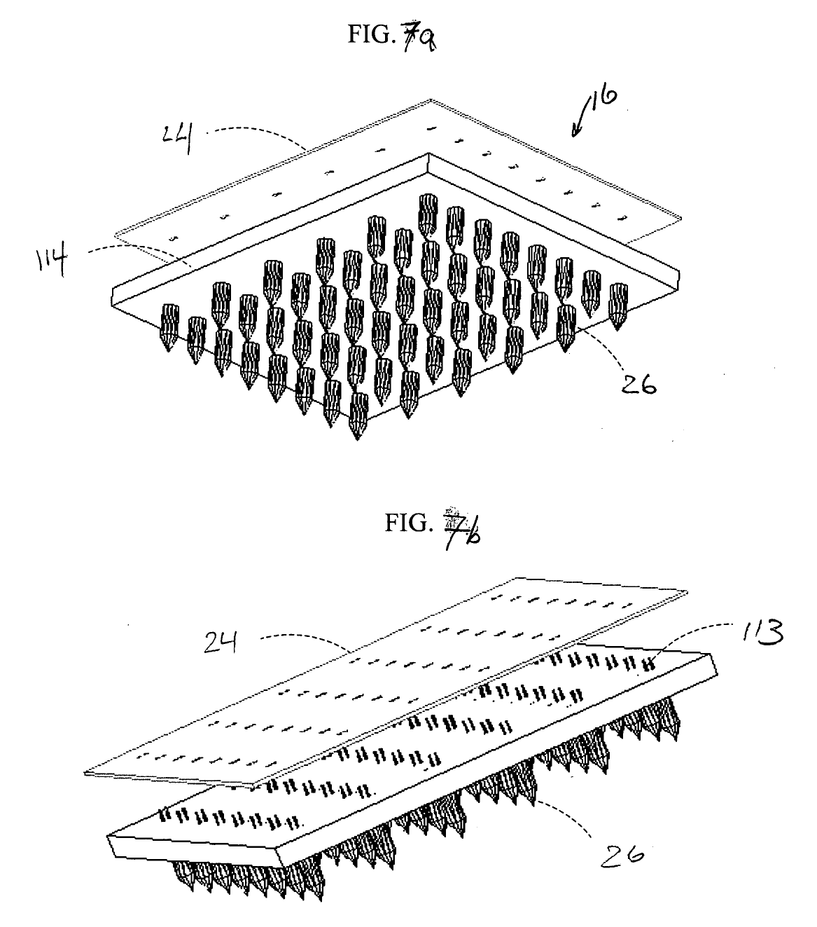

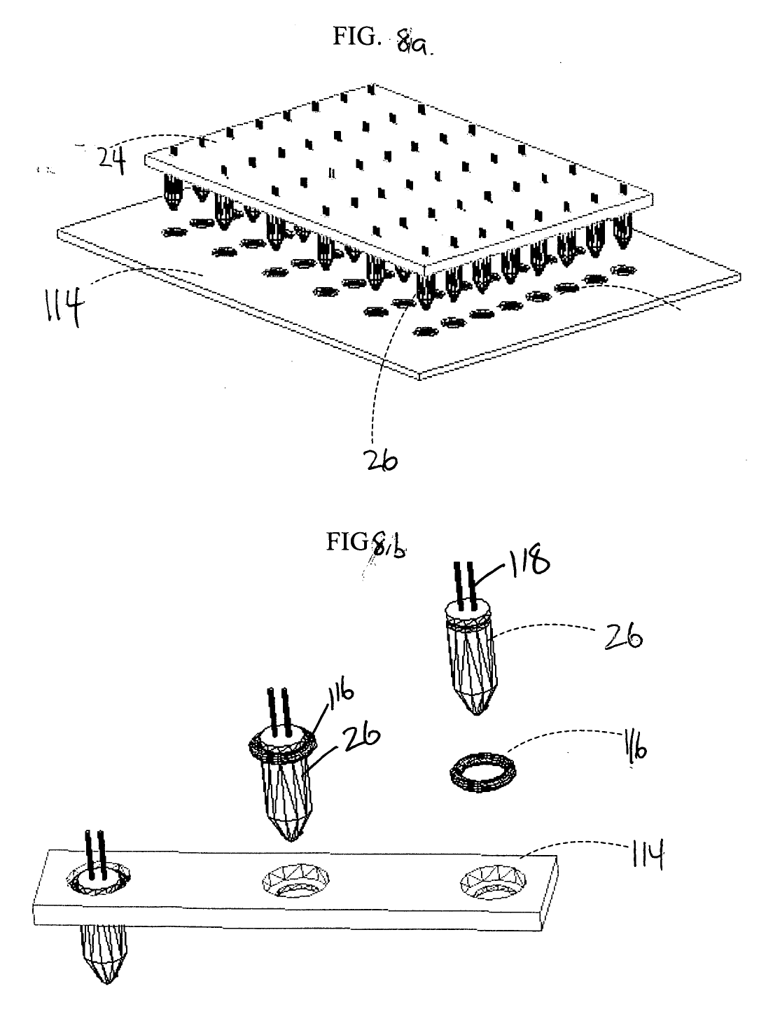

FIG. 9 shows is a more detailed diagram of an embodiment of an electrode. The electrode 26 comprises a protruding stud 130, at least one electrode 132 and electrode leads 134 housed within a sensor array base 114. In order to establish electrical contact between electrodes, the sensor array 16 is inserted into multiple test samples simultaneously. The electrodes are designed to minimize the potential of bubble formation during testing. For this reason, only non-wetting and insulting materials are used to enclose the electrodes. FIGS. 9a to 9d provide various stud shapes for use with the electrode. Each of these stud shapes, assist in reducing or eliminating the entrapment of bubbles at the indicator electrode(s) 132. All of these embodiments (cone, dome etc.) can be used for any of the embodiments of the sensor array.

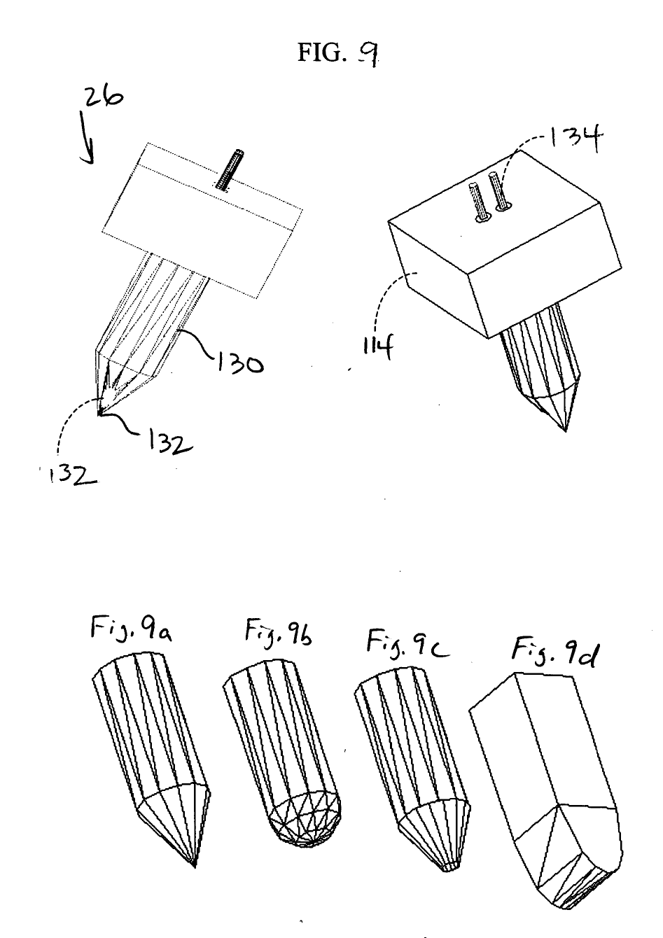

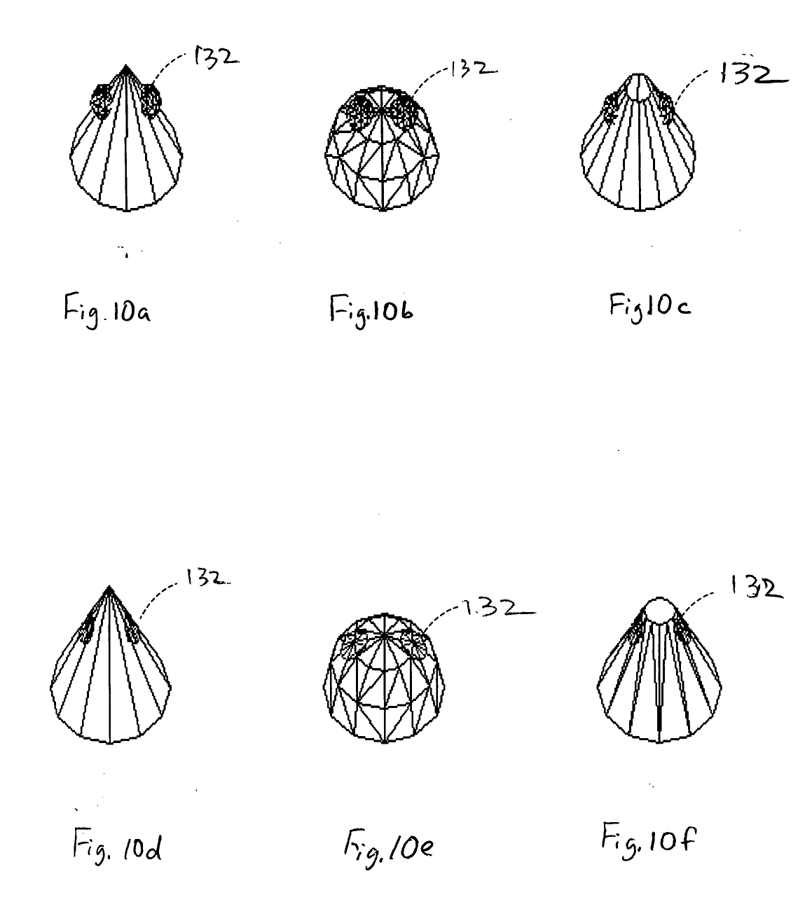

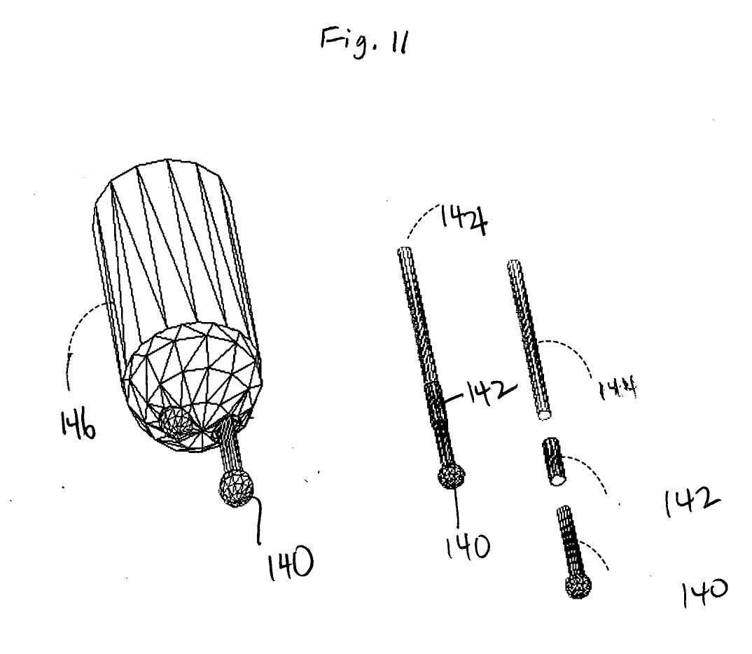

FIGS. 10a to 10f show various shapes and sizes of individual indicator electrode configurations. In FIG. 10a, two identical electrodes are spaced apart at a predefined distance while located near the tip of an electrode in order to facilitate an optimum electrical configuration. Established electric paths during the measurement are maintained through the close proximity of both electrodes 132. FIGS. 10a to 10c illustrate the application of three-dimensional electrode designs (spheres) using various tip shapes and sizes. This modular electrode configuration allows the application of various electrode sizes using the same sensor array by simply applying electrochemical cells that feature electrodes with increased diameters. FIGS. 10d to 10f show a planar electrode design where both electrodes 132 are located flush with the insulating tip. The shape and tip configuration of these electrodes are designed to minimize bubble formation and maximize bubble evasion during long measurement times since the occurrence of micro-bubbles can interfere with precision results.

FIG. 11 are schematic diagrams of another embodiment of an electrode. In this embodiment, the electrode 26 comprises a spherical platinum indicator electrode 140 mounted to a stainless steel sleeve 142 in order to establish electrical connection to the copper wire lead 144. The electrode configuration comprising the indicator electrode, sleeve and copper wire is inserted into pre-drilled openings of the insulating stud 146. In an alternative embodiment, the sleeve may be omitted and the lead wires 144 where the electrodes are inserted into the insulator studs directly connected to the electronics board or PCB.

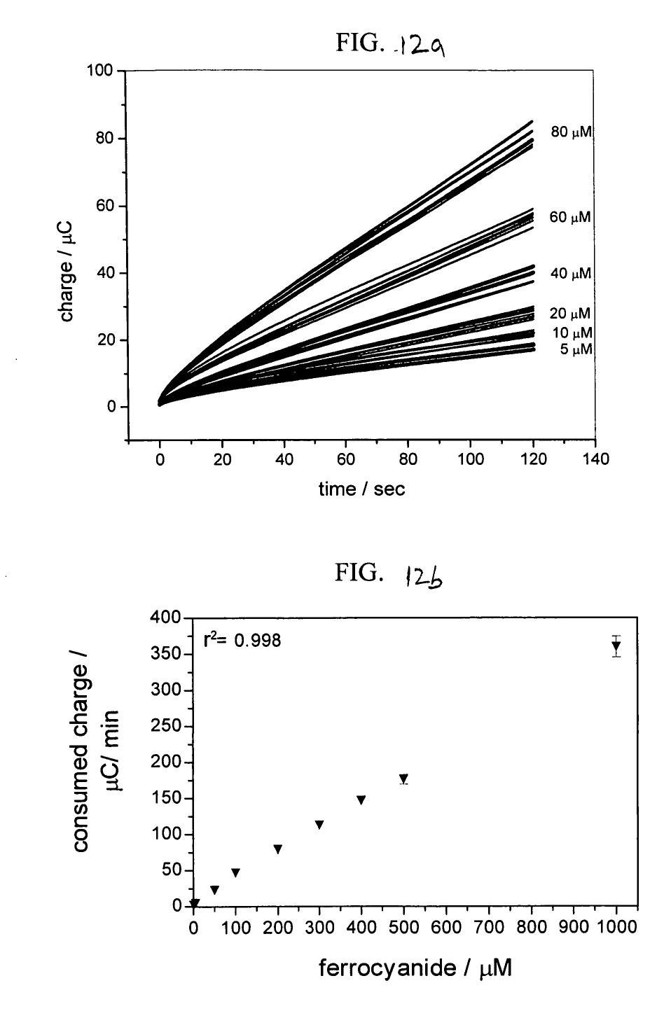

FIGS. 12a and 12b shows raw data obtained with a sensor array displayed using increasing concentrations of ferrocyanide as the reagent. In the displayed example, a solution containing the redox-couple ferricyanide (oxidized form) and ferrocyanide (reduced form) is prepared and added to the 48 wells. The ferricyanide concentration is set at 40 mM while increasing concentrations of ferrocyanide are added to each column containing eight electrochemical cells. During the measurement step, the reduced form is reconverted to the oxidized form at the anode and the magnitude of the measured current/charge is proportional to the ferrocyanide concentration in the test sample. Each electrochemical cell contained 250 μL of test solution and a constant voltage of 100 mV was applied between the two electrodes immersed in each well over a period of 120 sec. The resulting current is integrated to present total charge as a function of time (see FIG. 12a). In this two electrode configuration both indicator electrodes are made of platinum with similar electrode areas (approximately 0.03 cm²). The graphs further illustrate 8 repetitive measurements of increasing concentrations of ferrocyanide (5-10-20-40-60 and 80 μM) in the presence of 40 mM ferricyanide conducted in parallel. Using a similar experimental set up, FIG. 12b shows the calculated averaged slope values (μC/min; n=48 for each conc.) or consumed charges ( [symbol] Q between 60 and 120 sec) for a wider range of ferrocyanide concentrations (5-50-100-200-300-400-500-1000 μM). The presented sensor array exhibited a linear range over three orders in magnitude with a total precision of <4% RSD (n=384).

In another embodiment, the instruction control 20 is responsible for controlling and monitoring (via the CPU 44) the various operations of the electronics board such as application and/or removal of voltage (or current). Also, as part of its system monitoring function, if a fault is detected, appropriate action is performed to ensure that faulty measurement data are not collected.

Alternatively, the data processing module 22 is responsible for collecting, storing and analyzing measured data. Although shown as part of an external PC 18, it will be understood that this module 22 may be executed by the on-board controller 44 to retrieve and process data from the ADC 40.

In another embodiment, the apparatus may include a communication subsystem responsible for communication between the sensor array 16 and the user interface 46. As shown, user interface 46 may be implemented on a separate computing device which functions as a platform for further analysis and interfaces via a communication protocol such as Serial, TCP/IP, wireless such as “bluetooth” or Universal Serial Bus (USB). In the preferred embodiment, a Serial Communications protocol is implemented. In another embodiment, communications via Ethernet using TCP/IP is contemplated, which allows communication between one or more connected systems. This configuration could be extended to allow the instrument to be accessible from a remote computer.

The user interface 46 is responsible for interfacing with the user, communicating with the communication subsystem, processing and storing data. It also allows for the adjustment of various operating parameters such as sampling rate, run time, voltage output and others.

In another embodiment, the electronics board 14 may not include the set of multiplexers and therefore, the signal conditioning means 36 is directly connected to the set of ADCs 40.

The electrodes may be manufactured from a variety of materials such as gold, platinum, silver and others as well as their combinations. Each electrode is made of a three-dimensional protrusions (studs) designed to minimize the potential of bubble formation as soon as contact with a solution is established. The “bubble evasion” design is beneficial so as to maintain electrical contact between the electrodes and the test solution. Consequently, the sensor array and its electrodes comprise high stability, non-wetting insulating material. Contact between the electrodes and the data acquisition or wire management board is established via low resistance leads such as platinum or copper wires (Pt or Cu). Furthermore the application of insulating Si-layers and non-corroding materials are applied to limit occurrence of corrosion between metal to metal contact points.

As will be understood, a biamperometric measurement method was used to demonstrate the practical application and versatility of the invention. In brief, biamperometry is a technique based on two identical polarized electrodes and carries the advantage of simple instrumentation layout and measuring conditions, high sensitivity, high selectivity, and a high signal-to-noise ratio, which is attributed to the small applied potential difference (usually <200 mV).

The above-described embodiments of the present invention are intended to be examples only. Alterations, modifications and variations may be effected to the particular embodiments by those of skill in the art without departing from the scope of the invention, which is defined solely by the claims appended hereto.

1. Apparatus for high-speed acquisition of electrochemical measurements from multiple biochemical or microbiological samples comprising:
an array of electrodes;
a voltage signal generator for said array of electrodes; and
means for collecting electrochemical measurements from said electrodes;
wherein when said electrodes are brought in contact with said multiple biochemical or microbiological samples, said voltage signal generator provides a voltage to each of said electrodes to produce said electrochemical measurements for said means for collecting to retrieve.

2. The apparatus of claim 1 wherein said means for collecting comprises:
a signal conditioning device, connected to each of said electrodes, for receiving said electrochemical measurements;
a set of multiplexers for coordinating said electrochemical measurements; and
a set of analog-to-digital converters for converting each of said electrochemical measurements to a digital equivalent.

3. The apparatus of claim 2 said voltage signal generator and said means for collecting are located on an electronics board.

4. The apparatus of claim 2 wherein said signal conditioning device comprises at least one of a gain or a filter.

5. The apparatus of claim 1 further comprising:
means for moving said set of array of electrodes to bring said electrodes into contact with said samples and to remove said electrodes from contact with said samples.

6. The apparatus of claim 3 wherein said electronics board further comprises a central processing unit (CPU) for receiving said digital equivalents.

7. The apparatus of claim 6 further comprising means for processing and analyzing said digital equivalents.

8. The apparatus of claim 7 wherein said means is a software application.

9. The apparatus of claim 8 wherein said software application is stored in a computer remote from said electronics board or on said electronics board.

10. The apparatus of claim 1 further comprising a power source.

11. The apparatus of claim 3 wherein said electronics board further comprises a board conditioning device for monitoring the status and operation of components on said electronics board.

12. The apparatus of claim 9 wherein said computer further comprises:
a user interface; and
means for providing instructions to said electronics board via said CPU.

13. The apparatus of claim 3 wherein said electronics board further comprises:
a digital-to-analog converter for generating a voltage reference for the electrodes.

14. The apparatus of claim 13 wherein said digital to analog converter further comprises a feedback mechanism for verifying that said voltage reference to said electrodes matches an expected value.

15. The apparatus of claim 1 further comprising:
means for adding a buffer to each of said samples before said samples are brought in contact with said electrochemical cells.

16. The apparatus of claim 1 further comprising:
means for adding a microbe to each of said samples before said samples are brought in contact with said electrochemical cells.

17. The apparatus of claim 1 further comprising:
means for adding a reagent to each of said samples before said samples are brought in contact with said electrochemical cells.

18. A method of obtaining electrochemical measurements from multiple biochemical or microbiological samples comprising the steps of:
generating a reference voltage;
applying said reference voltage to a plurality of electrodes; and
retrieving electrochemical measurements from said electrodes after said plurality of electrodes contact said multiple samples.

19. The method of claim 18 further comprising the steps of:
converting said electrochemical measurements to digital equivalents.

20. The method of claim 19 further comprising the step of processing said digital equivalents.

21. The method of claim 19 further comprising the step, occurring before the step of converting, of:
signal conditioning said electrochemical measurements.

22. The method of claim 21 wherein said step of signal conditioning comprises the step of:
adding a gain to said electrochemical measurements.

23. The method of claim 21 wherein said step of signal conditioning comprises the step of:
filtering said electrochemical measurements.

24. The method of claim 19 comprising the step, before said step of converting, of:
multiplexing said electrochemical measurements.

25. The method of claim 18 further comprising the steps, occurring before said step of generating a voltage, of:
adding a buffer to said samples;
adding a microbe to said samples; and
adding a reagent to said samples.

26. The method of claim 25 further comprising the step, occurring after said step of adding a microbe, of:
incubating said mix of said buffer, microbe and sample.

27. The method of claim 26 further comprising the step, occurring after said step of adding a reagent, of:
incubating said mix of said buffer, microbe, reagent and sample.
